(19) United States
(12) Patent Application Publication
(10) Pub. No.: US 2011/0118428 A1
(43) Pub. Date: May 19, 2011

(54) FLUOROSURFACTANTS

(75) Inventors: Wolfgang Hierse, Gross-Zimmern (DE); Eckhard Claus, Frankfurt am Main (DE); Melanie Kleineidam, Darmstadt (DE); Peer Kirsch, Yokohama Kanagawa (JP); Elvira Montenegro, Weinheim (DE); Martin Seidel, Darmstadt (DE)

(73) Assignee: Merck Patent Gesellschaft Mit Beschrankterhaftung, Darmstadt (DE)

(21) Appl. No.: 13/003,185

(22) PCT Filed: Jun. 27, 2009
(86) PCT No.: PCT/EP09/04665

§ 371 Date: Jan. 7, 2011

(30) Foreign Application Priority Data
Jul. 7, 2008 (DE) 10 2008 031 599.0

(51) Int. Cl. C08F 16/26; C07C 317/46; C07C 315/00; C07C 69/734

(52) U.S. Cl. 526/247; 560/151; 560/181

(57) ABSTRACT

The present invention relates to novel compounds containing CF₃O end groups, to the use thereof as surface-active substances, and to processes for the preparation of these compounds.

The present invention relates to novel compounds containing CF₃O end groups, to the use thereof as surface-active substances, and to processes for the preparation of these compounds.

Fluorosurfactants have an outstanding ability to reduce surface tension, which is utilised, for example, in the hydrophobicisation of surfaces, for example of textiles, paper, glass, building materials or adsorbents. In addition, it is possible to use them as interface promoter or emulsifier or viscosity reducer in paints, surface coatings or adhesives.

In general, fluorosurfactants contain perfluoroalkyl substituents, which are degraded to perfluoroalkylcarboxylic acids (PFCAs) and -sulfonic acids (PFASs) in the environment by biological and/or other oxidation processes. In recent years, the accumulation of perfluoroalkylcarboxylic acids (PFCAs) and perfluoroalkylsulfonic acids (PFASs) in nature has given cause for concern. PFCAs and PFASs are highly persistent compounds whose long-chain variants (containing perfluoroalkyl chains of 8 or more carbon atoms) have a bioaccumulative potential. They are in some cases suspected of causing health problems (G. L. Kennedy, Jr., J. L. Butenhoff, G. W. Olsen, J. C. O'Connor, A. M. Seacat, R. G. Biegel, S. R. Murphy, D. G. Farrar, Critical Review in Toxicology, 2004, 34, 351-384).

Sulfosuccinates and/or sulfotricarballylates containing various fluorinated side chains are described in U.S. Pat. No. 4,968,599, U.S. Pat. No. 4,988,610 and U.S. Pat. No. 6,890,608 and in A. R. Pitt et al., Colloids and Surfaces A: Physicochemical and Engineering Aspects, 1996, 114, 321-335; A. R. Pitt, Progr. Colloid Polym. Sci., 1997, 103, 307-317 and Z.-T. Liu et al., Ind. Eng. Chem. Res. 2007, 46, 22-28.

The Omnova company markets polymers whose side chains contain terminal CF₃ or C₂F₅ groups. International Patent Application WO 03/010128 describes perfluoroalkyl-substituted amines, acids, amino acids and thio-ether acids which contain a C3-20-perfluoroalkyl group.

JP-A-2001/133984 discloses surface-active compounds containing perfluoroalkoxy chains which are suitable for use in antireflection coatings. JP-A-09/111286 discloses the use of perfluoropolyether surfactants in emulsions.

The earlier German Patent Application DE 102005000858 describes compounds which carry at least one terminal pentafluorosulfuranyl group or at least one terminal trifluoromethoxy group and contain a polar end group, are surface-active and are suitable as surfactants.

There continues to be a demand for alternative surface-active substances, preferably having a property profile comparable to that of classical fluoro-surfactants and equally great chemical versatility, which are preferably not degraded to long-chain persistent fluorocarboxylic or fluorosulfonic acids on oxidative or reductive degradation or are preferably effective as conventional fluorosurfactants in relatively low dosage.

Novel compounds have now been found which are suitable as surface-active substances and preferably do not have one or more of the above-mentioned disadvantages.

The present invention relates firstly to compounds of the formula (I)

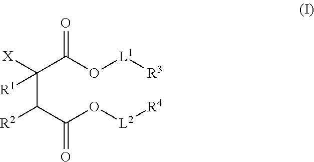

where

R¹ and R²=independently of one another, hydrogen or —CH₂—COO-L³—OCF₃,

R³ and R⁴=independently of one another, hydrogen or OCF₃, preferably both equal to OCF₃,

L¹, L² and L³=independently of one another, linear or branched alkyl, where one or more non-adjacent C atoms may be replaced by O, S and/or N, and/or one or more double and/or triple bonds and/or one or more CF₃O groups may be present in the chain and/or side chain, L¹, L² and L³ are preferably identical, and

X=an anionic polar group,

and the compounds contain at least one CF₃O group.

The compounds according to the invention may contain one or more trifluoromethoxy groups, where the CF₃O groups may be bonded to the carboxylate groups as R³ and/or R⁴ and/or as substituents of L¹, L² and/or L³. Compounds containing one to six trifluoromethoxy groups, in particular those containing two or three trifluoromethoxy groups, are preferred. In a preferred variant of the invention, R³ and/or R⁴ are equal to CF₃O groups and are bonded to the carboxyl groups via linear alkyl chains which contain no further CF₃O groups. In another preferred variant of the invention, R³ and/or R⁴ are equal to CF₃O groups and are bonded to the carboxyl groups via branched alkyl chains which contain no further CF₃O groups.

L¹, L² and L³ are preferably, independently of one another, linear or branched alkyl having 1 to 20 C atoms, preferably 1 to 10 C atoms. In particular, L¹, L² and L³ are, independently of one another, linear alkyl having 3 to 10 C atoms, particularly preferably having 3 to 8 C atoms. In a preferred variant of the invention, L¹ and L² are identical. If L³ is also present, L¹ and L² or L¹ and L³ or L² and L³ can preferably be identical. Particularly preferred compounds are those in which L¹=L²=L³.

Preferred compounds are, in particular, those compounds in which all variables have the preferred meanings.

The compounds according to the invention are preferably based on esters of succinic acid and tricarballylic acid, where the compounds contain at least one CF₃O group.

In a preferred group of compounds of the formula I, R¹ and R² stand for hydrogen and R³ and R⁴ stand for a CF₃O group. These compounds are represented by formula (Ia):

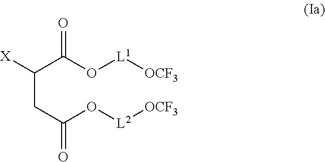

In another preferred group of compounds of the formula I, R¹ stands for H, R² stands for —CH₂—COO-L³-OCF₃ and R³ and R⁴ stand for a CF₃O group. These compounds are represented by formula (Ib):

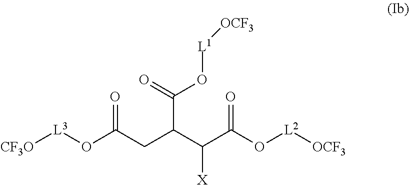

In a further preferred group of compounds of the formula I, R¹ stands for —CH₂—COO-L³-OCF₃, R² stands for hydrogen and R³ and R⁴ stand for a CF₃O group. These compounds are represented by formula (Ic):

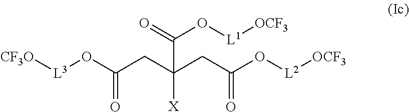

The anionic compounds of the formula I or the compounds of the formula I which can be converted into anionic salts preferably contain, as counterion, an alkali metal ion, preferably Li⁺, Na⁺ or K⁺, an alkaline-earth metal ion or NR₄⁺, where R═H⁺ or C1-C6-alkyl and all R may be identical or different, preferably NH₄⁺ or also triethylammonium.

In a preferred group of compounds to be employed in accordance with the invention or compounds according to the invention, X stands for an anionic polar group selected from —COOM, —SO₃M, —OSO₃M, —PO₃M₂, —OPO₃M₂, —(OCH₂CH₂)ₛ—O—(CH₂)ₜ—COOM, —(OCH₂CH₂)ₛ—O—(CH₂)ₜ—SO₃M, —(OCH₂CH₂)ₛ—O—(CH₂)ₜ—OSO₃M, —(OCH₂CH₂)ₛ—O—(CH₂)ₜ—PO₃M₂, —(OCH₂CH₂)ₛ—O—(CH₂)ₜ—OPO₃M₂ or for the formulae A to C:

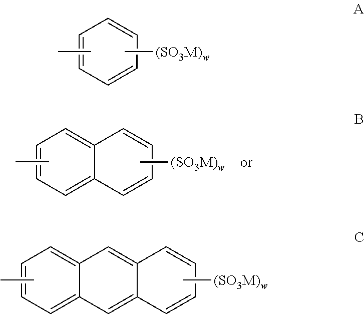

where M stands for H or an alkali metal ion, preferably Li⁺, Na⁺ or K⁺, or NH₄⁺, in particular Na⁺, s stands for an integer from the range from 1 to 1000, t stands for an integer selected from 1, 2, 3 or 4 and w stands for an integer selected from 1, 2 or 3.

The preferred anionic groups here include, in particular, —COOM, —SO₃M, —OSO₃M, —PO₃M₂, —OPO₃M₂, the sub-formula A, and —(OCH₂CH₂)ₛ—O—(CH₂)ₜ—COOM, —(OCH₂CH₂)ₛ—O—(CH₂)ₜ—SO₃M and —(OCH₂CH₂)ₛ—O—(CH₂)ₜ—OSO₃M, where each individual one of these groups taken for itself may be preferred.

The very particularly preferred anionic groups here include —SO₃M, —OSO₃M, —PO₃M₂ and OPO₃M₂.

A preferred variant of the invention are sulfosuccinates which contain one or two trifluoromethoxyalkyl groups and sulfotricarballylates which contain one, two or three trifluoromethoxyalkyl groups. In the present invention, the term sulfosuccinates or sulfotricarballylates is applied to esters, preferably di- or triesters, of maleic or aconitic acid to the double bond of which a sulfonate group has been added. Sulfosuccinates containing two trifluoromethoxy groups and sulfotricarballylates containing three trifluoromethoxy groups are particularly preferred.

Particularly preferred compounds according to the invention are compounds of the formulae (II), (III) and (IV):

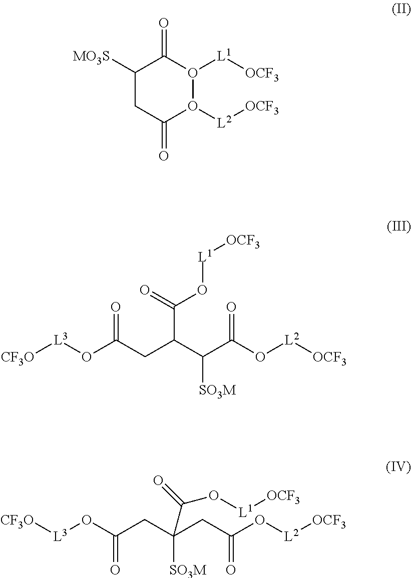

M and L¹, L² and L³ have the general and preferred meanings mentioned for formula (I). L¹, L² and L³ are preferably, independently of one another, linear alkyl having 3 to 10 C atoms, in particular having 3 to 8 C atoms, and M is a monovalent cation. Particular preference is given to compounds of the formulae (II), (III) and (IV) in which all L are identical.

M is preferably ═H⁺, an alkali metal cation or NR₄⁺, where R═H⁺ or C1-C6-alkyl and all R may be identical or different. X is particularly preferably ═Na⁺, K⁺ or NH₄⁺, particularly preferably Na⁺.

L¹, L² and L³ are preferably, independently of one another, equal to linear C3-C10-alkyl, in particular linear C3-C8-alkyl. L¹ and L² are particularly preferably, independently of one another, equal to linear C5-C10-alkyl for compounds of the formula (II). For compounds of the formulae (III) and (IV), L¹, L² and L³ are preferably, independently of one another, equal to linear C3-C6-alkyl. Particular preference is given to compounds in which L¹, L² and L³ contain no further CF₃O groups.

In another variant of the invention, L¹, L² and L³ can be, independently of one another, branched alkyl groups and/or contain further CF₃O groups.

Particular preference is given to compounds in which both X and also L¹, L² and L³ have the preferred meanings.

Examples of particularly preferred compounds according to the invention are:

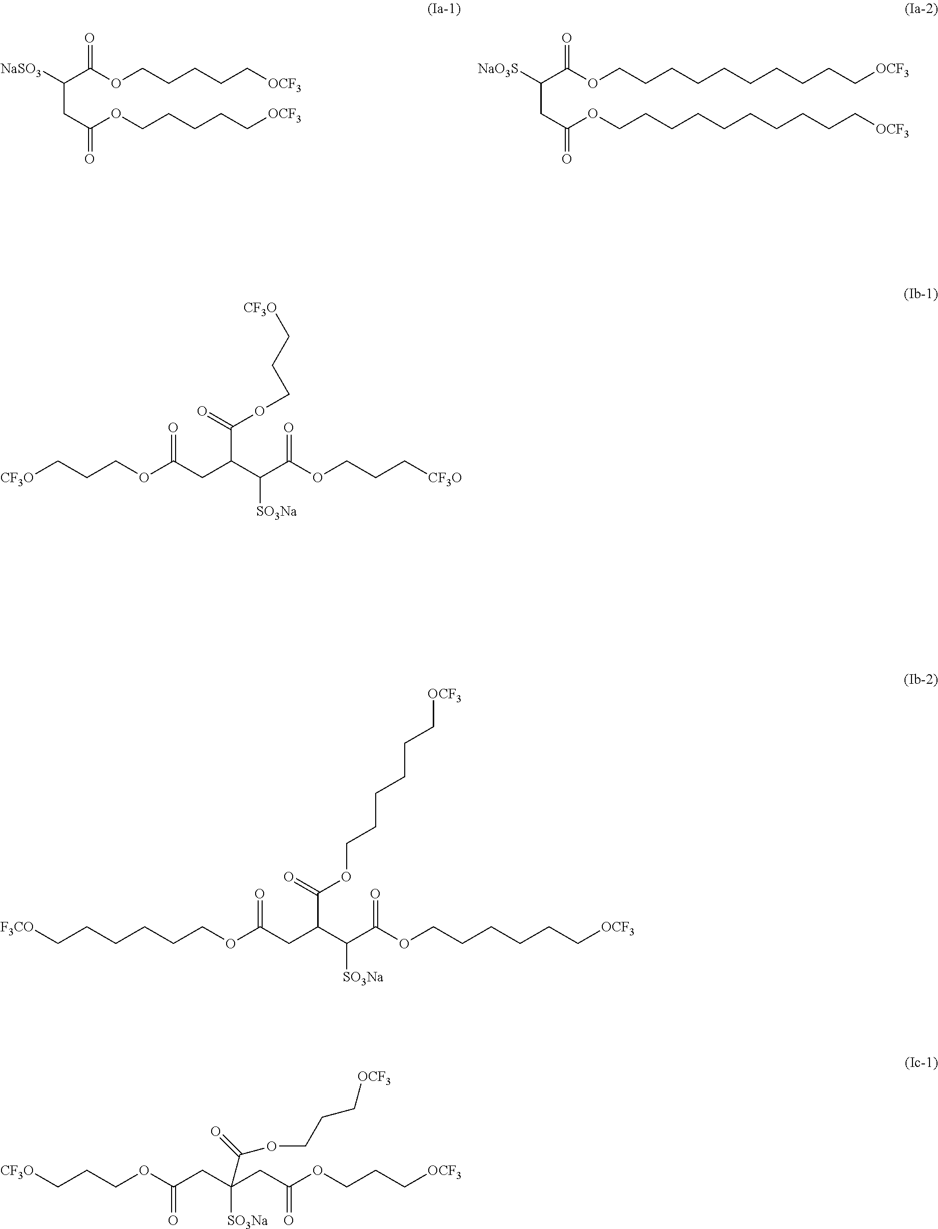

The compounds of the formulae (I) to (IV) according to the invention may also be in the form of isomer mixtures (constitutional and/or configurational isomer mixtures). In particular, diastereomer and/or enantiomer mixtures are possible.

Advantages of the compounds according to the invention may be, in particular:
a surface activity which is equal or superior to that of conventional hydrocarbon surfactants with respect to efficiency and/or effectiveness,
biological and/or abiotic degradability of the substances without the formation of persistent perfluorinated degradation products, such as PFOA (perfluorooctanoic acid) or PFOS (perfluorooctanesulfonate),
weak foam formation,
good processability in formulations and/or
storage stability.

The compounds according to the invention preferably have particular surface activity.

The present invention relates secondly to the use of compounds of the formulae (I), (II) and/or (III) as surface-active agents, for example for improving the flow behaviour and wetting ability of coating formulations.

Preference is given to the use of compounds of the formulae (II) and/or (III). The preferred embodiments of the compounds according to the invention described above can particularly advantageously be used here. Sulfosuccinates which contain one or two, in particular two, trifluoromethoxyalkyl groups and sulfotricarballylates which contain one, two or three, in particular three, trifluoromethoxyalkyl groups are preferably used. The compounds of the formulae (I) to (IV) according to the invention can also be used as isomer mixtures (constitutional and/or configurational isomer mixtures). In particular, diastereomer and/or enantiomer mixtures are possible.

Areas of application are, for example, the use of the compounds according to the invention as additives in surface-coating preparations, such as paints, coatings, protective coatings, speciality coatings in electronic or semiconductor applications (for example photoresists, top antireflective coatings, bottom antireflective coatings) or in optical applications (for example photographic coatings, coatings of optical elements) or in additive preparations for addition to corresponding preparations.

For use, the compounds according to the invention are usually incorporated into correspondingly designed compositions. The present invention likewise relates to corresponding preparations comprising at least one compound according to the invention. Such compositions preferably comprise a vehicle which is suitable for the particular application and optionally further active substances and/or optionally assistants. Preferred compositions here are paint and surface-coating preparations and printing inks.

In addition, the present invention also relates to water-based surface-coating formulations which comprise at least one of the compounds according to the invention, alone or mixed with other surfactants. Preference is given to the use of surface-coating formulations based on the following synthetic film formers:
polycondensation resins, such as alkyd resins, saturated/unsaturated polyesters,
polyamides/imides, silicone resins; phenolic resins; urea resins and melamine resins,
polyaddition resins, such as polyurethanes and epoxy resins,
polymerisation resins, such as polyolefins, polyvinyl compounds and polyacrylates.

In addition, the compounds according to the invention are also suitable for use in surface coatings based on natural products and modified natural products. Preference is given to surface coatings based on oils, polysaccharides, such as starch and cellulose, and also based on natural resins, such as cyclic oligoterpenes, polyterpenes and/or shellac.

The compounds according to the invention can be used both in physically hardening (thermoplastics) and in crosslinking (elastomers and thermosets) aqueous surface-coating systems.

The compounds according to the invention preferably improve the flow and wetting properties of the surface-coating systems.

A further use of the compounds according the invention is the use in the synthesis of polymers, especially fluorinated polymers. Important industrial methods for the preparation of fluoropolymers, such as, for example, polytetrafluoroethylene (PTFE), are, for example, emulsion and suspension polymerisation. Suspension and emulsion polymerisation processes are standard polymerisation processes which are well known to the person skilled in the art. In suspension and emulsion polymerisation processes, the system always comprises at least four constituents: (predominantly) water-insoluble monomer, water, dispersant or emulsifier and initiator. The performance of the said polymerisation processes is familiar to the person skilled in the art. In these processes, the polymer is prepared in an autoclave which contains water, the corresponding, usually gaseous, monomer(s), initiator(s), surfactant(s) and other assistant(s), with stirring and constant temperature and pressure control. The compounds according to the invention are suitable as surfactants to keep the very hydrophobic fluoropolymer droplets or particles dispersed in the aqueous solution.

Furthermore, the compounds of the invention can be used in hydrophobicising agents, oleophobicising agents, wetting agents, flow-control agents, protection/cleaning agents against spots and soiling, stain releases, antifogging agents, lubricants, antifoamers, deaerating agents, drying accelerators, as abrasion-resistance and mechanical wear-resistance enhancers, and antistatics, particularly in the treatment of textiles (in particular clothing, carpets and carpeting, upholstery in furniture and automobiles) and hard surfaces (in particular kitchen surfaces, sanitary installations, tiles, glass), non-woven textile materials, leather goods, papers and cardboard articles, wood and wood-based materials, mineral substrates, such as stone, cement, concrete, plaster, ceramics (glazed and unglazed tiles, earthenware, porcelain) and glasses, and for plastics and metallic substrates. For metallic substrates, the present invention additionally also relates to the use of compounds according to the invention in anticorrosion agents. The present invention furthermore also relates to the use thereof as mould-release agents in plastics processing. In the case of cleaning compositions and spot removers, the use as detergent or dirt emulsifier and dispersant is additionally also an advantageous embodiment of the present invention.

Surfactants according to the invention are furthermore also suitable as antimicrobial active compound, in particular as reagents for antimicrobial surface modification.

In printing inks, the compounds according to the invention can likewise advantageously be employed and have one or more of the following functions: antifoam, deaerating agent, friction-control agent, wetting agent, flow-control agent, pigment-compatibility enhancer, print-resolution enhancer, drying accelerator.

Compounds according to the invention can be employed as foam stabiliser and/or for supporting film formation, in particular in aqueous film-forming fire-extinguishing foams, both synthetic and also protein-based, and also for alcohol-resistant formulations (AFFF and AFFF-AR, FP, FFFP and FFFP-AR fire-extinguishing foams).

The compounds according to the invention can also advantageously be used as additives in polymeric materials (plastics) with one or more of the following functions: lubricant, internal-friction reducer, UV stabiliser, hydrophobicising agent, oleophobicising agent, protection agent against spots and soiling, coupling agent for fillers, flame retardant, migration inhibitor (in particular against migration of plasticisers), antifogging agent.

Furthermore, the compounds according to the invention can be used as additives in liquid media for cleaning, etching, reactive modification and/or substance deposition on metal surfaces (in particular also electroplating and anodisation) or semiconductor surfaces (in particular for semiconductor photolithography: developer, stripper, edge bead remover, etching and cleaning composition), as wetting agent and/or deposited film quality enhancer.

In addition, the compounds which can be used in accordance with the invention as surfactants are suitable for washing and cleaning applications as well as for use as additives/surfactants in cosmetic products, such as, for example, hair- and bodycare products (shampoos, hair rinses and hair conditioners), foam baths, creams and lotions with one one or more of the following functions: emulsifiers, wetting agent, foaming agent, lubricant, antistatic, skin-grease resistance enhancer.

Compounds according to the invention act as additives in herbicides, pesticides and fungicides, with one or more of the following functions: substrate wetting agent, adjuvant, foam inhibitor, dispersant, emulsion stabiliser.

In addition, they can be employed as additives in de-icing agents or icing inhibitors.

In addition, the compounds of the invention can be used as additives in preparations for ore processing, in particular flotation and leaching solutions, with one or more of the following functions: wetting agent, foaming agent, foam inhibitor, as well as additives in agents for the stimulation of oil wells, with one or more of the following functions: wetting agent, foaming agent, emulsifier.

Compounds according to the invention can be employed as additives in adhesives, with one or more of the following functions: wetting agent, penetration agent, substrate adhesion enhancer, antifoam.

Compounds according to the invention can also serve as additives in lubricants and hydraulic fluids, with one or more of the following functions: wetting agent, corrosion inhibitor. In the case of lubricants, the use as dispersant (in particular for fluoropolymer particles) is additionally also an essential aspect.

On use as additives in putty and filling compositions, compounds according to the invention can act with one or more of the following functions: hydrophobicising agent, oleophobicising agent, protection agent against soiling, weathering-resistance enhancer, UV stabiliser, silicone bleeding inhibitor.

The present invention relates to all uses mentioned here of compounds to be employed in accordance with the invention. The respective use of surfactants for the said purposes is known to the person skilled in the art, and consequently the use of the compounds to be employed in accordance with the invention presents no problems. Preferably compounds of formula (I) are used in paints and surface coatings and in the synthesis of polymers, especially fluorinated polymers.

The compounds according to the invention can preferably be prepared by esterification of maleic acid and aconitic acid or anhydrides or acid chlorides thereof using one or more alcohols of the formula (V)

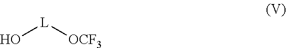

and subsequent addition reaction, preferably of sodium hydrogensulfite. L in formula (V) and in formulae (VI) to (IX) below has the meaning described for L¹, L² and L³ in formula (I), in particular also the preferred meanings. The alcohols of the formula (V) may contain one or more CF₃O groups, preferably one CF₃O group.

The present invention thus relates thirdly to a process for the preparation of compounds of the formula (I), in particular sulfosuccinates which contain one or two trifluoromethoxyalkyl groups and sulfotricarballylates which contain one, two or three trifluoromethoxyalkyl groups.

The synthesis of the alcohols of the formula (V) is known from the literature and is described in WO 2006/072401.

The succinates are preferably synthesised in the presence of a conventional catalyst, such as, for example, toluene-4-sulfonic acid monohydrate:

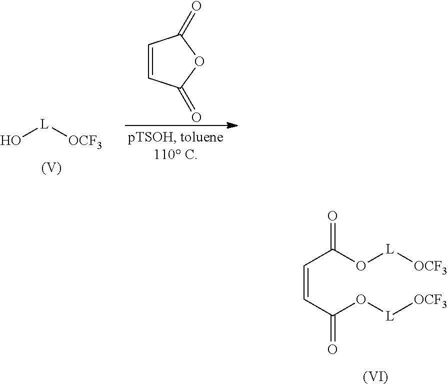

In a second step, the group X is introduced by addition onto the double bond. The following scheme shows by way of example the synthesis of sulfosuccinates by the addition reaction of sodium hydrogensulfite, which can be carried out under conditions known to the person skilled in the art:

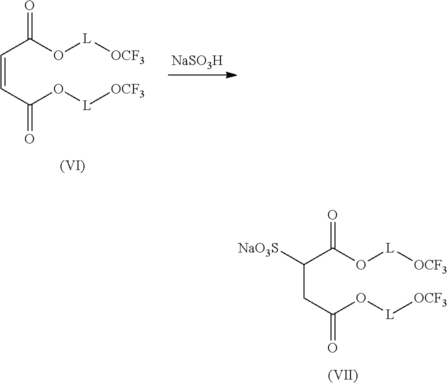

The synthesis of aconitic esters is preferably carried out in the presence of a conventional catalyst, such as, for example, toluene-4-sulfonic acid monohydrate:

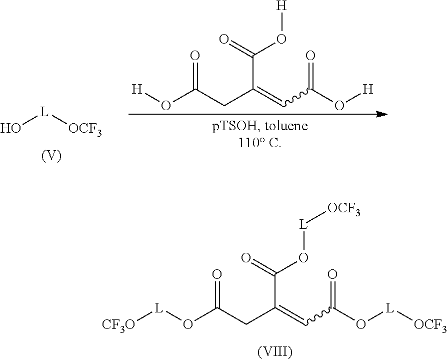

In a second step, the group X is then introduced by addition onto the double bond. The following scheme shows by way of example the synthesis of sulfotricarballylates by the addition reaction of sodium hydrogensulfite, which can be carried out under conditions known to the person skilled in the art:

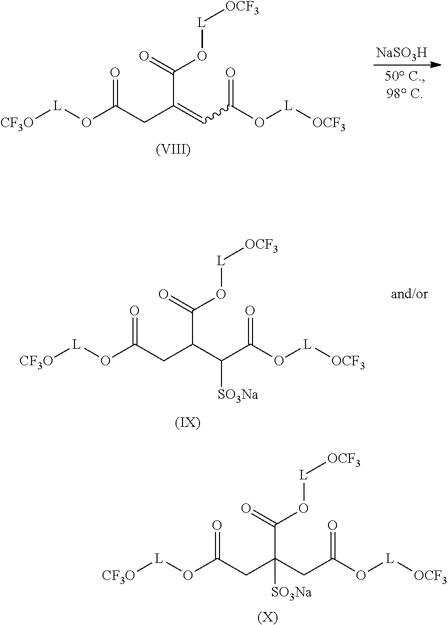

Formula (VIII) shows the presence of Z/E double-bond isomers. The preparation of further compounds according to the invention can be carried out analogously to the illustrative reactions shown above. The preparation of further compounds according to the invention can also be carried out by other methods known per se to the person skilled in the art from the literature. In particular, other esterification catalysts can be used.

The present invention relates fourthly to compounds of the formulae (VI) and (VIII) which occur as intermediates in the above-described syntheses of the compounds of the formula (I) according to the invention, in particular the sulfosuccinates and sulfotricarballylates:

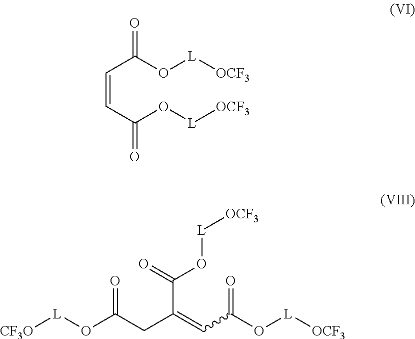

where L has the meaning described above and preferred embodiments.

The present invention relates fifthly to the use of the compounds of the formulae (VI) and (VIII) as monomers or comonomers in the synthesis of fluorinated polymers according to the processes described above.

Apart from the preferred compounds mentioned in the description, the use thereof, compositions and processes, further preferred combinations of the subject-matters according to the invention are disclosed in the claims.

The disclosures in the cited references thus expressly also belong to the disclosure content of the present application.

The following examples explain the present invention in greater detail without restricting the scope of protection. In particular, the features, properties and advantages described in the examples of the compounds and reaction conditions on which the relevant examples are based can also be applied to other substances and compounds which are not mentioned in detail, but fall within the scope of protection, unless stated otherwise elsewhere. In addition, the invention can be carried out throughout the range claimed and is not restricted to the examples mentioned here.

### EXAMPLES

### Example 1

### Synthesis of the Sulfosuccinate of the Formula (la-1)

a) Esterification

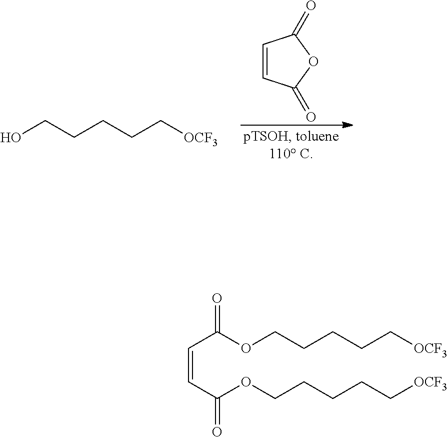

A mixture of 6.9 g (22.49 mmol) of trifluoromethoxy alcohol, 0.75 g (7.5 mmol) of maleic anhydride and 0.26 g (1.5 mmol) of toluene-4-sulfonic acid monohydrate in 12 ml of dichloromethane is stirred under reflux for 15 hours. The water liberated during the reaction is removed with the aid of a water separator. The mixture is quenched using water and subsequently extracted with toluene, and the combined organic phases are washed with water, dried over sodium sulfate and filtered. The solvent is distilled off in a rotary evaporator.

Purification: by column chromatography over silica gel

Eluent: dichloromethane/heptane 9/1

Substance: C₁₆H₂₂F₆O₆; M=424.333 g/mol

¹H-NMR (300 MHz): 6.25 (s); 4.2 (t); 3.97 (t); 1.79-1.67 (m); 1.57-1.43 (m) ppm.

¹³C-NMR (300 MHz): 165.22; 129.78; 126.69/123.33/119.97/116.61; 67.13/67.09; 64.86; 28.25; 27.88; 21.99 ppm.

¹⁹F-NMR (280 MHz): −60.77 (s)ppm.

b) Addition Reaction

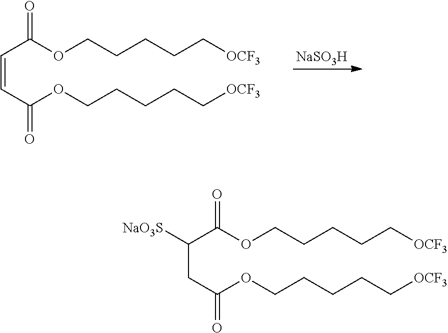

64.8 ml (325 mmol) of sodium hydrogensulfite (39% solution in water) are added at 50° C. to a solution of 5.5 g (12.96 mmol) of diester in 44 ml of 1,4-dioxane, and the mixture is stirred under reflux for 10-15 hours. The reaction temperature is subsequently held at 98° C. for 30-40 hours. The solvent is distilled off in a rotary evaporator.

Purification: recrystallisation from ethyl acetate

Substance: C₁₆H₂₃F₆O₉S*Na; M=528.39 g/mol

¹H-NMR (400 MHz): 4.27-4.20 (m); 4.18 (2×d); 4.14-4.06 (m); 4.02 (t); 3.98 (t); 3.15 (2×d); 3.05 (2×d); 1.80-1.60 (m); 1.54-1.37 (m) ppm.

¹³C-NMR (400 MHz, D₂O): 171.81; 168.81; 120.41; 67.60; 67.30; 66.25; 65.38; 61.94; 33.41; 27.85; 27.50; 27.38; 21.54; 21.44 ppm.

¹⁹F-NMR (380 MHz, D₂O): −60.87 (s); −61.18 (s) ppm.

### Example 2

### Synthesis of the Sulfotricarballylate of the Formula (Ib-1)

a) Esterification

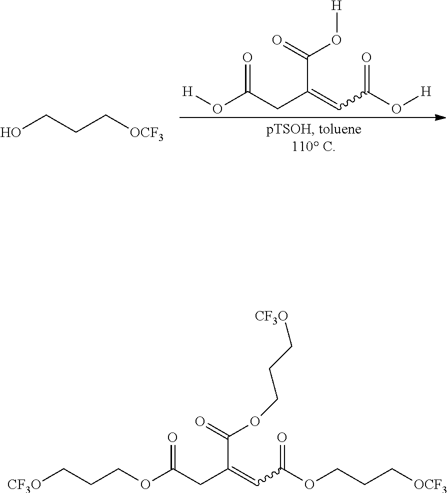

A mixture of 63.25 g (95.69 mmol) of trifluoromethoxy alcohol, 3.4 g (19.14 mmol) of aconitic acid (cis, 98%, Alfa Aesar) and 0.66 g (3.83 mmol) of toluene-4-sulfonic acid monohydrate in 40 ml of toluene is stirred under reflux for 15 hours. The water liberated during the reaction is removed with the aid of a water separator. The mixture is quenched using water. The mixture is subsequently extracted with toluene, and the combined organic phases are washed with water, dried over sodium sulfate and filtered. The solvent is distilled off in a rotary evaporator.

Purification: by column chromatography over silica gel

Eluent: toluene/ethyl acetate 1/5

Substance: C₁₈H₂₁F₉O₉; M=552.34 g/mol

¹H-NMR (400 MHz): 6.88 (s); 4.3-4.09 (m); 3.86 (s); 2.1-1.92 (m) ppm.

¹³C-NMR (300 MHz): 168.99/165.23/164.56; 139.23; 128.50; 126.27/122.92/119.57/116.21; 64.95/64.91/64.72/64.68; 61.80/60.91/60.50; 32.67; 27.41/27.29 ppm.

¹⁹F-NMR (280 MHz): −59.40/−59.45/−59.51 (m) ppm.

MS (70 eV) m/e: 552 (M⁺, 8%); 409 (100%).

b) Addition Reaction

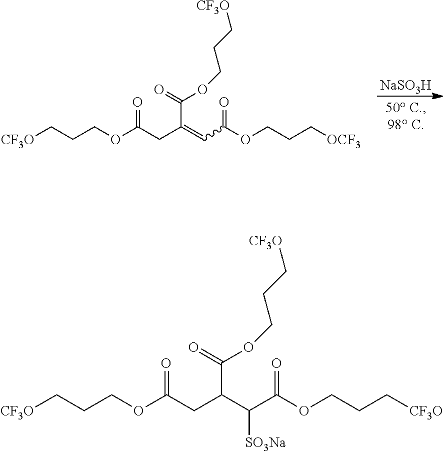

89.38 ml (449 mmol) of sodium hydrogensulfite (39% solution in water) are added at 50° C. to a solution of 10.2 g (18.47 mmol) of the triester in 44 ml of 1,4-dioxane, and the mixture is stirred under reflux for 10-15 hours. The reaction temperature is subsequently held at 98° C. for 30-40 hours. The solvent is distilled off in a rotary evaporator.

Purification: recrystallisation from methanol

Substance: C₁₈H₂₂F₉O₁₂S*Na; M=656.401 g/mol

¹H-NMR (400 MHz): 4.25-3.99 (m); 3.89 (d); 3.79 (d); 3.50-3.43 (m); 3.25-3.22 (m); 3.21-3.19 (m); 3.13-3.10 (m); 3.04 (2×d); 2.83 (2×d); 2.02-1.87 (m) ppm.

¹³C-NMR (300 MHz): 172.14; 171.83; 171.38; 168.52; 167.42; 126.31/122.95/119.59/116.24; 65.08/64.91; 64.33; 60.37/60.00/59.82; 41.23; 40.91; 32.89; 31.63; 27.61/27.55/27.46 ppm.

¹⁹F-NMR (280 MHz): −59.94/−59.03/−59.14/−59.20/−59.22/−59.23 (m) ppm.

MS (70 eV) m/e: 679 (M⁺+2×Na⁺, 14%); 593 (100%).

### Example 3

### Synthesis of the Sulfosuccinate of the Formula (Ia-2)

a) Esterification

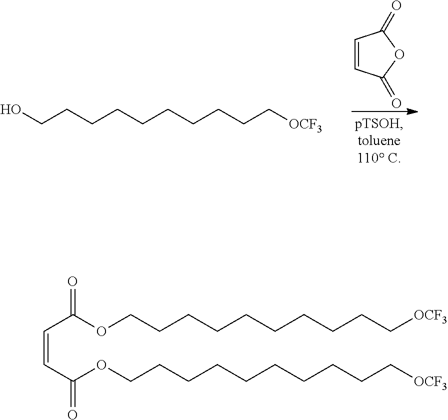

A mixture of 7.02 g (28.93 mmol) of 10-trifluoromethoxypentan-1-ol, 1 g (9.99 mmol) of maleic anhydride and 0.34 g (1.99 mmol) of toluene-4-sulfonic acid monohydrate in 21 ml of toluene is stirred under reflux for 15 hours. The water liberated during the reaction is removed with the aid of a water separator. The reaction is subsequently terminated by addition of 50 ml of deionised water, extracted with toluene, and the combined organic phases are washed with water, dried over sodium sulfate and filtered. The solvent is distilled off in a rotary evaporator.

Substance: C₂₆H₄₂F₆O₆; M=564.598 g/mol

¹H-NMR (400 MHz, DMSO-d₆): 6.66 (s); 6.36 (s); 4.06 (t); 4.03-3.89 (m);1.59-1.45 (m), 1.29-1.13 (m) ppm.

¹⁹F-NMR (380 MHz, DMSO-d₆): −58.84 (s)ppm.

b) Addition Reaction

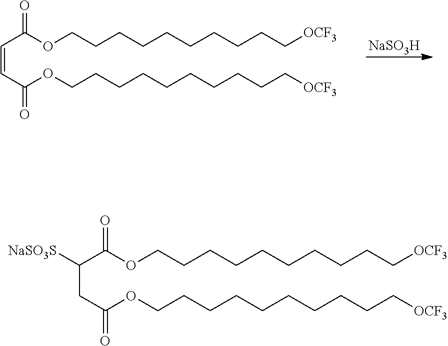

47.8 ml (240 mmol) of sodium hydrogensulfite (39% solution in water) are added at 50° C. to a solution of 5.4 g (9.56 mmol) of bis(trifluoromethoxydecyl) (Z)-but-2-enediote in 33 ml of 1,4-dioxane, and the mixture is stirred under reflux for 10-15 hours. The reaction temperature is subsequently held at 98° C. for 30-40 hours. The solvent is distilled off in a rotary evaporator.

Purification: recrystallisation from diethyl ether

Substance: C₂₆H₄₃F₆O₉SNa; M=668.66 g/mol

¹H-NMR (400 MHz, DMSO-d₆): 4.04 (t); 3.96 (m); 3.60 (dd); 2.97 (dd); 2.85 (dd); 1.63 (quin); 1.56-1.46 (m); 1.38-1.21 (m) ppm.

¹³C-NMR (300 MHz, DMSO-d₆): 171.00; 168.39; 123.02/119.67; 68.18; 64.01; 61.35; 34.03; 28.75; 28.56; 28.35; 28.04; 25.21; 24.77 ppm.

¹⁹F-NMR (380 MHz, DMSO-d₆): −59.65 (s)ppm.

MS (FIA-ESI, 70 eV) m/e: pos. mode: [M+2Na⁺]⁺=691; neg. mode: [M⁻]=645.

### Example 4

### Synthesis of the Sulfotricarballylate of the Formula (Ib-2)

a) Esterification

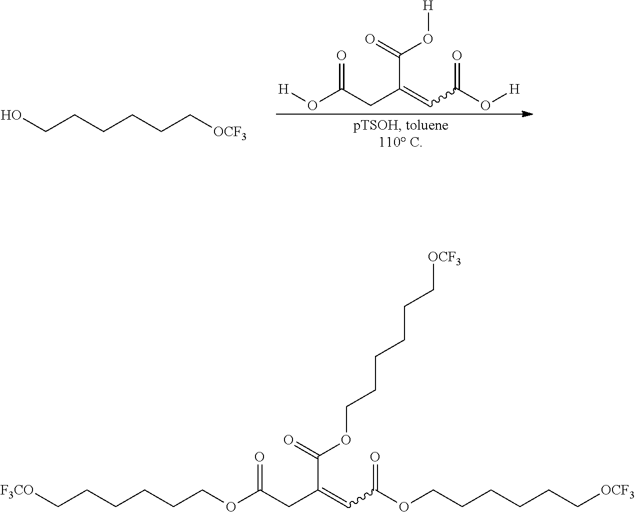

1.60 g (9.01 mmol) of cis-aconitic acid and 29.47 mg (0.17 mmol) of toluene-4-sulfonic acid monohydrate are added to 8.06 g (43.29 mmol) of 6-trifluoromethoxyhexan-1-ol in 29 ml of toluene, and the mixture is stirred at 110° C. for 15 hours. The water liberated during the reaction is removed with the aid of a water separator. The reaction is subsequently terminated by addition of 20 ml of deionised water, extracted with toluene, and the combined organic phases are washed with water and dried over sodium sulfate and filtered. The solvent is distilled off in a rotary evaporator.

Substance: C₂₇H₃₉F₉O₉; M=678.579 g/mol

¹H-NMR (400 MHz, DMSO-d₆): 6.81 (s); 4.19-4.108 (m); 4.10-3.99 (m); 3.84 (s); 1.71-1.51 (m); 1.46-1.26 (m) ppm.

¹³C-NMR (300 MHz, DMSO-d₆): 169.06/165.36/164.72; 139.40; 128.84/128.35/128.14; 126.36/125.25/123.01/119.66; 68.03; 65.51/65.39/64.62/64.28/60.51; 32.79; 32.25; 29.83/28.10/27.89/27.77/27.66; 24.90/24.69/24.38 ppm.

¹⁹F-NMR (380 MHz, DMSO-d₆): −58.66 to −58.97 (m) ppm.

MS (70 eV) m/e: 678 (M⁺, 13%); 55 (100%).

b) Addition Reaction

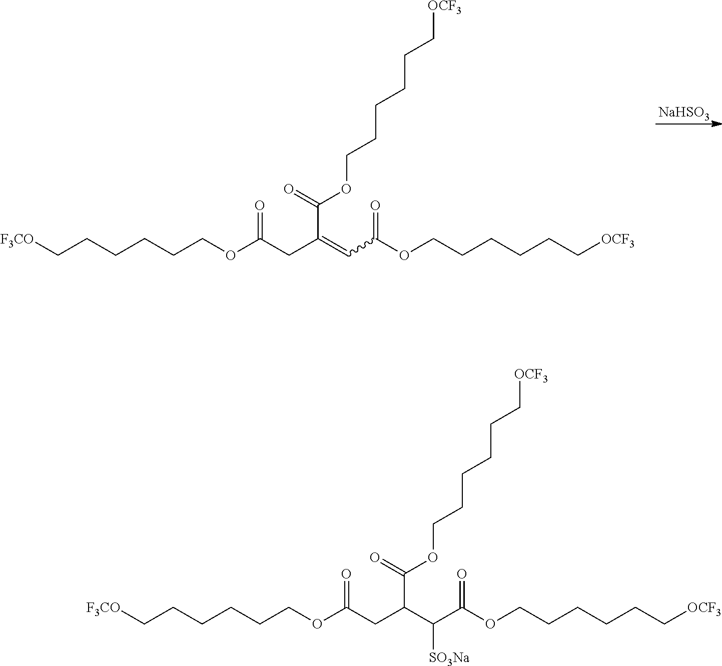

25.5 ml (128 mmol) of sodium hydrogensulfite (39% solution in water) are added at 50° C. to a solution of 4.72 g (5.29 mmol) of 3-trifluoromethoxy-hexyl (Z)-3-(3-trifluoromethoxyhexoxycarbonyl)pent-2-enedioate in 18 ml of dioxane, and the mixture is stirred under reflux for 10-15 hours. The reaction temperature is subsequently held at 98° C. for 30-40 hours. The solvent is distilled off in a rotary evaporator.

Purification: recrystallisation from diethyl ether

¹H-NMR (400 MHz, DMSO-d₆): 4.05 (t); 4.01-3.91 (m); 3.85 (d); 3.71 (d); 3.47 (ddd); 3.32 (ddd); 3.19 (dd); 3.11 (dd); 2.95 (dd); 2.79 (dd); 1.67-1.57 (m); 1.56-1.44 (m); 1.37-1.23 (m) ppm.

¹³C-NMR (100 MHz, DMSO-d₆): 172.34; 172.25; 172.18; 171.43; 168.53; 167.38; 121.31; 68.15; 64.86-63.43; 41.59; 41.06; 33.36; 31.32; 28.01-27.83; 24.81-24.77; 24.71-24.47 ppm.

¹⁹F-NMR (380 MHz, DMSO-d₆): −59.55 (s)ppm.

MS (FIA-ESI, 70 eV) m/e: pos. mode: [M+H]⁺=783; neg. mode: [M−Na]⁻=759.

### Example 5

### Determination of the Static Surface Tension

The static surface tensions γ of aqueous surfactant solutions having various concentrations (weight percentages) are determined.

Instrument: Sinterface tensiometer (model PAT1)

Temperature of the measurement solutions: room temperature

Measurement method employed: measurement of the surface tension on hanging droplets against air. The principal radii of curvature (r₁ and r₂) of the ellipsoids (droplets) are determined here by droplet contour analysis. Since the pressure difference (Ap) between the outside and inside of an interface is indirectly proportional to the radii of curvature, the surface tension can be calculated from the following correlation:

$$Δ p = γ ( \frac{1}{{r}_{1}} + \frac{1}{{r}_{2}} )$$

Instrument settings: droplet volumes 7-10 mm³; measurement time 1500-3600 s; 1.5 pictures/s, droplet density=1 g/cm³

### Example 5a

Table 1 shows the static surface tension y of an aqueous solution of the sulfosuccinate according to Example 1b) as a function of the concentration c.

TABLE 1

| c [%] | γ [mN/m] |
|---|---|
| 0.0010 | 69.1 |
| 0.0125 | 56.0 |
| 0.0241 | 52.9 |
| 0.0482 | 46.5 |
| 0.0963 | 40.6 |
| 0.1926 | 34.0 |
| 0.4815 | 27.6 |
| 0.9630 | 25.2 |
| 0.9630 | 25.2 |
| 1.0400 | 25.6 |
| 1.0593 | 25.8 |
| 1.9260 | 24.6 |

### Example 5b

Table 2 shows the static surface tension γ of an aqueous solution of the sulfotricarballylate according to Example 2b) as a function of the concentration c.

TABLE 2

| c [%] | γ [mN/m] |
|---|---|
| 0.0001 | 75.8 |
| 0.00028 | 67.9 |
| 0.00098 | 64.9 |
| 0.003 | 59.3 |
| 0.01 | 50.7 |
| 0.03 | 43.7 |
| 0.1 | 37.3 |
| 0.3 | 30.6 |
| 0.497 | 27.2 |
| 0.696 | 25.5 |
| 1 | 24.6 |
| 2 | 23.4 |

### Example 5c

Table 3 shows the static surface tension γ of an aqueous solution of the sulfosuccinate according to Example 3b) as a function of the concentration c.

TABLE 3

| c [%] | γ [mN/m] |
|---|---|
| 0.0001 | 67.4 |
| 0.0007 | 30.2 |
| 0.0009 | 26.7 |
| 0.0010 | 26.1 |
| 0.0013 | 25.9 |
| 0.0024 | 24.8 |
| 0.0049 | 24.6 |
| 0.0098 | 24.5 |
| 0.0997 | 24.8 |
| 1.0000 | 24.5 |

### Example 5d

Table 4 shows the static surface tension γ of an aqueous solution of the sulfotricarballylate according to Example 4b) as a function of the concentration c.

TABLE 4

| c [%] | γ [mN/m] |
|---|---|
| 0.0001207 | 68.4 |
| 0.00135 | 30.8 |
| 0.001551 | 30.3 |
| 0.00236 | 27.1 |
| 0.00343 | 26.0 |
| 0.00409 | 25.5 |
| 0.00462 | 25.5 |
| 0.0139 | 24.2 |
| 0.119 | 23.8 |
| 1.00 | 23.7 |

### Example 6

### Determination of the Dynamic Surface Tension

The dynamic surface tensions 7 of 0.1% (weight percentage) aqueous solutions of compounds 2b), 3b) and 4b) are determined.

Measurement method employed: measurement of the surface tension using the bubble pressure method

Instrument: SITA tensiometer (model t 60)

Temperature of the measurement solutions: 26° C.±0.2° C. (Example 6c);

21° C.±0.1° C. (Examples 6a and 6b)

In the measurement of the dynamic surface tension, air bubbles are forced through a capillary into the surfactant solution at different rates. From the resultant pressure change, the surface tension can be determined as a function of the bubble life using the following equation:

$$γ = \frac{r ( {p}_{max} - ρ \cdot g \cdot h )}{2}$$

pₘₐₓ=maximum pressure, ρ=density of the liquid, h=immersion depth, r=radius of the capillary

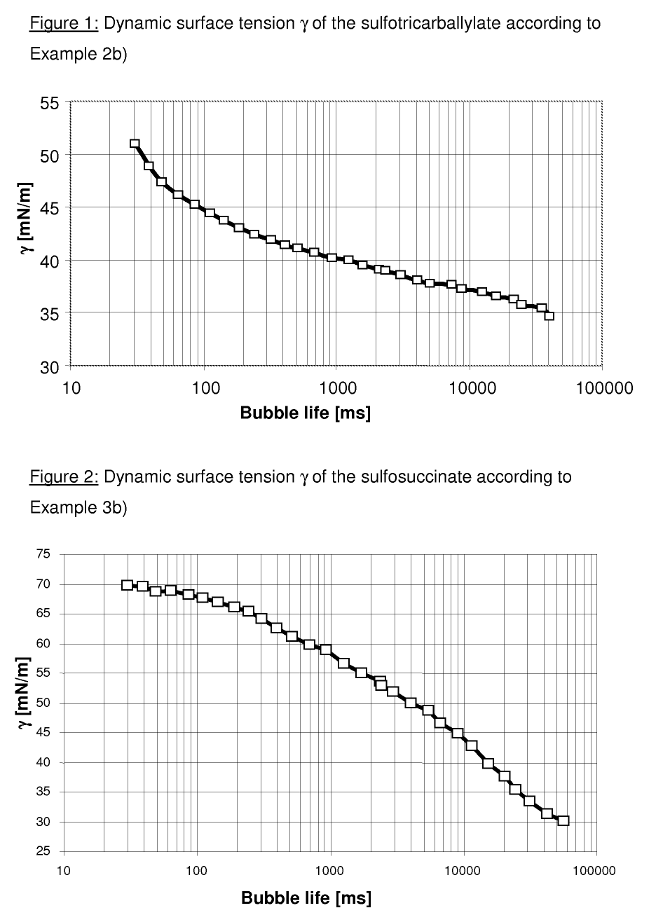

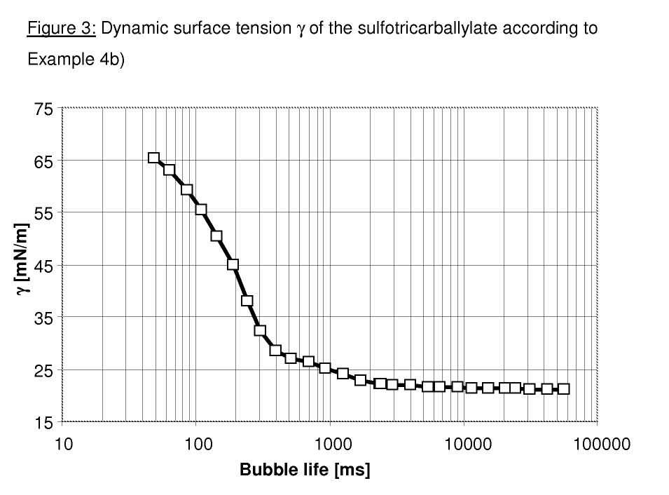

The measurement values are shown in Tables 5-7. FIGS. 1-3 show the dynamic surface tensions as a function of the bubble life for compounds 2b), 3b) and 4b).

### Example 6a

Table 5 shows the dynamic surface tension γ of a 0.1% aqueous solution of the sulfotricarballylate according to Example 2b) as a function of the bubble life.

TABLE 5

| Bubble life [ms] | γ [mN/m] |
|---|---|
| 31 | 51 |
| 39 | 48.9 |
| 49 | 47.3 |
| 64 | 46.1 |
| 84 | 45.2 |
| 111 | 44.4 |
| 146 | 43.7 |
| 187 | 43 |
| 243 | 42.4 |
| 320 | 41.9 |
| 422 | 41.4 |
| 554 | 41.1 |
| 670 | 40.7 |
| 931 | 40.2 |
| 1286 | 40 |
| 1712 | 39.5 |
| 2283 | 39.1 |
| 2549 | 39 |
| 3286 | 38.6 |
| 4318 | 38.1 |
| 5435 | 37.8 |
| 7082 | 37.7 |
| 8922 | 37.3 |
| 12142 | 37 |
| 15363 | 36.6 |
| 19657 | 36.2 |
| 26417 | 35.7 |
| 32593 | 35.4 |
| 42559 | 34.6 |

### Example 6b

Table 6 shows the dynamic surface tension γ of a 0.1% aqueous solution of the sulfosuccinate according to Example 3b) as a function of the bubble life.

TABLE 6

| Bubble life [ms] | γ [mN/m] |
|---|---|
| 30 | 69.7 |
| 39 | 69.6 |
| 49 | 68.7 |
| 64 | 68.8 |
| 86 | 68.1 |
| 110 | 67.6 |
| 144 | 66.9 |
| 191 | 66 |
| 243 | 65.4 |
| 307 | 64.1 |
| 393 | 62.6 |
| 515 | 61.2 |
| 701 | 59.7 |
| 936 | 58.9 |
| 1253 | 56.5 |
| 1702 | 55 |
| 2376 | 53.6 |
| 2404 | 52.9 |
| 2997 | 51.8 |
| 4025 | 49.9 |
| 5426 | 48.6 |
| 6747 | 46.6 |
| 9023 | 44.8 |
| 11682 | 42.7 |
| 15432 | 39.8 |
| 20542 | 37.6 |
| 24501 | 35.3 |
| 31642 | 33.5 |
| 42712 | 31.4 |
| 56145 | 30.1 |

### Example 6c

Table 7 shows the dynamic surface tension γ of a 0.1% aqueous solution of the sulfotricarballylate according to Example 4b) as a function of the bubble life.

TABLE 7

| Bubble life [ms] | γ [mN/m] |
|---|---|
| 49 | 65.3 |
| 64 | 63 |
| 86 | 59.3 |
| 110 | 55.4 |
| 144 | 50.4 |
| 191 | 44.9 |
| 243 | 38 |
| 307 | 32.3 |
| 393 | 28.5 |
| 515 | 27.1 |
| 701 | 26.3 |
| 936 | 25.2 |
| 1253 | 24 |
| 1702 | 22.7 |
| 2376 | 22.2 |
| 2404 | 22.2 |
| 2997 | 21.9 |
| 4025 | 21.9 |
| 5426 | 21.6 |
| 6747 | 21.6 |
| 9023 | 21.5 |
| 11682 | 21.4 |
| 15432 | 21.3 |
| 20542 | 21.3 |
| 24501 | 21.3 |
| 31642 | 21.2 |
| 42712 | 21.1 |
| 56145 | 21.2 |

### Example 7

### Antipitting Test in a Polyurethane Water-Borne Surface Coating

A surface coating is prepared from the raw materials according to Table 8 in which surface defects (pits) are generated specifically by overdosing the antifoam BYK 023. A highly concentrated solution (39.05% by weight) of the sulfotricarballylate according to Example 2b in Dowanol PM (Dow Chemicals) is incorporated into the surface coating in various amounts and compared in efficacy to the 0 sample (without sulfotricarballylate).

TABLE 8
Raw materials for surface-coating formulation

| Product | Function | Manufacturer |
|---|---|---|
| Bayhydrol XP | Binder | Bayer |
| 2470 | | |
| Bayhydur 304 | PUR curing agent | Bayer |
| Methoxybutyl | Solvent | VWR |
| acetate | | |
| BYK-023 | Antifoam | BYK Chemie |
| Blue pigment | Contrast to substrate | BASF |
| paste | | |

In the preparation of the contaminated surface-coating sample, firstly binder, pigment paste and water are initially introduced (Table 9a; PE beaker: 860 ml), and some of the initially introduced mixture is subsequently completed (Table 9b; PE beaker 350 ml).

TABLE 9a

| Initially introduced components 1 | | | |
|---|---|---|---|
| | | Formulation | Dissolver/stirrer |
| Pos. | Raw material | [g] | disc setting |
| 1 | Bayhydrol XP 2470 | 271.18 | 400 rpm; |
| 2 | Pigment paste | 8 | 10 min; 80 mm |
| 3 | H₂O | 27.28 | |

TABLE 9b

| Completion | | | |
|---|---|---|---|
| | | Formulation | Dissolver/stirrer |
| Pos. | Raw material | [g] | disc setting |
| 4 | Initially introduced | 161.00 | 400 rpm; |
| | mixture | | 10 min; 80 mm |
| 5 | Bayhydur 304 | 51.39 | |
| 6 | Methoxybutyl | 12.87 | |
| | acetate | | |
| 7 | H₂O | 24.57 | |
| 8 | BYK 023 | 0.17 | 600 rpm; |
| | | | 10 min; 60 mm |

For incorporation of the surfactant solution, the latter is firstly initially introduced (PE beaker, 175 ml), and the amount of surface coating is subsequently based on the weight of surfactant. Three test coatings having a different surfactant content or without surfactant are prepared (Table 10).

TABLE 10

Incorporation of the surfactant solution

| | Weight of | Weight of | | |
|---|---|---|---|---|
| Surface- | surfactant | surface | C* | Dissolver/ |
| coating | solution | coating | surfactant | stirrer disc |
| sample | [g] | [g] | [%] | setting |
| 1 | — | — | 0 sample | — |
| 2 | 38.3973 | 0.0447 | 0.045 | 900 rpm; |
| | | | | 5 min; |
| | | | | 40 mm |
| 3 | 30.5281 | 0.1066 | 0.136 | 900 rpm; |
| | | | | 5 min; |
| | | | | 40 mm |

*Surfactant content based on the total amount of surface-coating sample

The surface coatings are applied to white paint cards (219×286 mm; manufacturer: Leneta) with the aid of an automatic film applicator (vacuum suction) with a film applicator frame (capacity: 4 ml of surface coating; 20 ml spraying; application rate: 50 mm/s; wet-layer thickness: 30 μm). Surface-coating sample 2 is virtually free of surface defects (pits) and surface-coating sample 3 is completely free of surface defects, whereas surface-coating sample 1 exhibits numerous surface defects (pits).

FIGS. 1-3 show the dynamic surface tensions as a function of the bubble life for compounds 2b), 3b) and 4b).

1. Compounds of the formula (I) 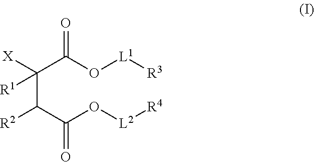
where
R¹ and R²=independently of one another, hydrogen or —CH₂—COO—L³-OCF₃,
R³ and R⁴=independently of one another, hydrogen or OCF₃,
L¹, L² and L³=independently of one another, linear or branched alkyl, where one or more non-adjacent C atoms may be replaced by O, S and/or N, and/or one or more double and/or triple bonds and/or one or more CF₃O groups may be present in the chain and/or side chain, and
X=an anionic polar group,
and the compounds contain at least one CF₃O group.

2. Compounds according to claim 1, characterised in that X is equal to —SO₃M, —OSO₃M, —PO₃M₂ or —OPO₃M₂, where M is an alkali metal ion, an alkaline-earth metal ion or NR₄⁺, where R═H⁺ or C1-C6-alkyl and all R may be identical or different.

3. Compounds according to claim 1, characterised in that the compounds contain two or three CF₃O groups.

4. Compounds according to claim 1, characterised in that L¹=L²=L³ and are equal to linear or branched alkyl having 1 to 20 C atoms.

5. Compounds according to claim 1, characterised in that R¹ and R² stand for hydrogen and R³ and R⁴ stand for a CF₃O group.

6. Compounds according to claim 1, characterised in that R¹ stands for H, R² stands for —CH₂—COO-L³-OCF₃ and R³ and R⁴ stand for a CF₃O group.

7. Compounds according to claim 1, characterised in that R¹ stands for —CH₂—COO-L³-OCF₃, R² stands for hydrogen and R³ and R⁴ stand for a CF₃O group.

8. Compounds according to claim 1, characterised in that L¹ and L² are, independently of one another, equal to linear C5-C10-alkyl.

9. Compounds according to claim 1, characterised in that L¹, L² and L³ are, independently of one another, equal to linear C3-C6-alkyl.

10. Compounds according to claim 1, characterised in that L¹, L² and L³ contain no further CF₃O groups.

11. Compounds according to claim 1, characterised in that they conform to the formula (II), (III) or (IV) 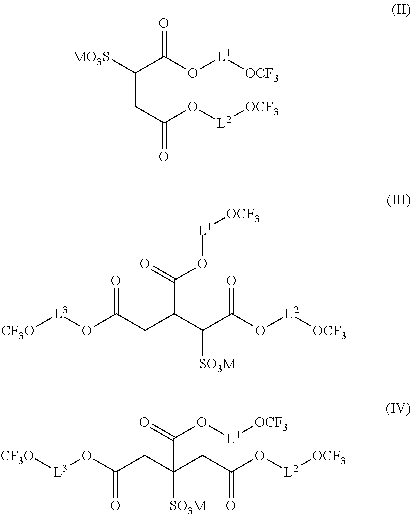
where
M=H or an alkali metal ion, preferably Li⁺, Na⁺ or K⁺, or NH₄⁺, and L¹, L² and L³=independently of one another, linear or branched alkyl, where one or more non-adjacent C atoms may be replaced by O, S and/or N, and/or one or more double and/or triple bonds and/or one or more CF₃O groups may be present in the chain and/or side chain.

12. A method of achieving a surface-active effect comprising a compound according to claim 1.

13. Process for the preparation of compounds according to claim 1 comprising a) the esterification of maleic acid and aconitic acid or anhydrides or acid chlorides thereof using one or more alcohols of the formula (V) 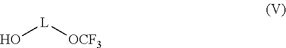
preferably in the presence of a catalyst, and b) the addition reaction, preferably of sodium hydrogensulfite, onto the olefinic double bond, where L has the meaning described for L¹, L² and L³ in formula (I), and the alcohols of the formula (V) contain one or more CF₃O groups.

14. Compounds of the formulae (VI) and (VIII): 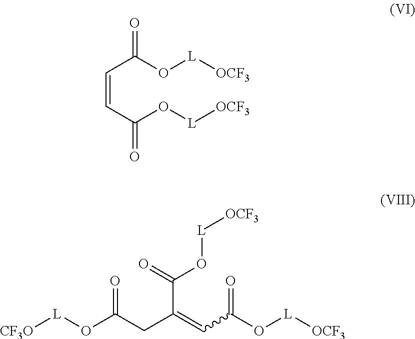
where L has the meaning described for L¹, L² and L³ in formula (I).

15. A method for the synthesis of fluorinated polymers comprising synthesizing using a compound of the formulae (VI) and (VIII) as monomer or comonomer.
